(19) United States
(12) Patent Application Publication
(10) Pub. No.: US 2017/0143207 A1
(43) Pub. Date: May 25, 2017

(54) Apparatus and method for detecting lesion in brain magnetic resonance image, and computer-readable recording medium for implementing the method

(71) Applicant: SUNGSHIN WOMEN'S UNIVERSITY INDUSTRY-ACADAMIC COOPERATION FOUNDATION, Seoul (KR)

(72) Inventors: Sudipta Roy, Hooghly (IN); Sanjay Nag, Kolkata (IN); Samir Kumar Bandyopadhyay, Kolkata (IN); Debnath Bhattacharyya, Pune, IN (US); Tai-Hoon Kim, Suwon-si (KR)

(73) Assignee: SUNGSHIN WOMEN'S UNIVERSITY INDUSTRY-ACADEMIC COOPERATION FOUNDATION, Seoul (KR)

(21) Appl. No.: 14/757,520

(22) Filed: Dec. 23, 2015

(30) Foreign Application Priority Data
Nov. 23, 2015 (KR) 10-2015-0163802

(51) Int. Cl. A61B 5/00; A61B 5/055

(52) U.S. Cl. CPC A61B 5/0042; A61B 5/055; A61B 5/726; A61B 5/4064

(57) ABSTRACT

Disclosed is an apparatus and method for detecting a brain lesion in a magnetic resonance (MR) image, and computer-readable recording medium for implementing the method. The apparatus includes an image area selector for receiving an MR image and creating an image of a brain portion (brain portion image) in which the brain portion is selectively presented; and an image processor for receiving the brain portion image and performing contrast adjustment on the brain portion image to obtain an image for diagnosis in which an area with a suspected lesion is emphasized.

### CROSS-REFERENCE TO RELATED APPLICATION

This application claims the benefit of Korean Patent Application No. 10-2015-0163802, filed on Nov. 23, 2015 in the Korean Intellectual Property Office, the disclosures of which are incorporated herein by reference.

### BACKGROUND OF THE INVENTION

1. Field of the Invention

The present disclosure relates to an apparatus and method for detecting a brain lesion in a magnetic resonance (MR) image, and a computer-readable recording medium for implementing the method, which detects the presence and location of a brain lesion by processing an MR image.

2. Description of the Related Art

A Magnetic Resonance Imaging (MRI) device obtains an image from a patient through computation of a computer in the device by putting the patient into a strong magnetic field, instantaneously emitting a radio frequency signal that only excites hydrogen nuclei (or protons) in a tissue of the patient, and delivering the radio frequency signal to the computer when, after the lapse of a predetermined period of time, during relaxation of the tissue, the excited hydrogen nuclei that had absorbed the radio frequency signal discharges the radio frequency signal. The intensity of the discharged signal varies depending on the quantity of the hydrogen atoms contained by each tissue and the tissue-specific T1 and T2 relaxation time. Accordingly, the MR image typically includes a T1 weighted image and a T2 weighted image that reflect a difference in T1 and T2 relaxation times between tissues.

To make a brain diagnosis through MR images obtained by the MR imaging device, the user should obtain an MR image that distinctly differentiates a lesion (or hemorrhage) portion from other portions, and for the distinction of the lesion portion, a technology to detect the lesion by issuing different weights to respective portions in the MR image has been used.

However, according to the lesion detection technology in the prior art as disclosed in Korean Patent No. 10-1203047, the user needs to select every reference image for analysis of an MR image, and repeat complicated settings in the analysis process, thereby creating a problem in that it requires a long time for the analysis, and that the accuracy of differentiation of the lesion portion may depend on the user's selection.

### SUMMARY OF THE INVENTION

Accordingly, the present disclosure has been made keeping in mind the above problems occurring in the prior art, and an object of the present invention is to provide an apparatus and method for detecting a brain lesion from a magnetic resonance image, and a computer-readable recording medium for implementing the method, by which to obtain an image for diagnosis that accurately represents a portion with a suspected lesion while minimizing user actions for settings, by performing filtering to eliminate portions other than a brain portion and performing power-law transform on pixel values within the brain portion.

In accordance with an aspect of the present disclosure, an apparatus for detecting a brain lesion in a magnetic resonance (MR) image is provided. The apparatus includes an image area selector for receiving an MR image and creating an image of a brain portion (brain portion image) in which the brain portion is selectively represented; and an image processor for receiving the brain portion image and performing contrast adjustment on the brain portion image to obtain an image for diagnosis in which an area with a suspected lesion is emphasized.

The image area selector may include a binarization transformer for receiving the MR image, and selecting pixels having signal intensities that exceed a standard deviation of signal intensities of all pixels within the MR image are selected for a brain portion; a wavelet transformer for wavelet-transforming the brain portion selected by the binarization transformer to create an image for adjustment; a quick-hull processor for processing the image for adjustment in a quick hull scheme to create a convex image for adjustment; and a convex processor for obtaining the brain portion image by applying the convex image for adjustment to the MR image.

The contrast adjustment may adjust the brain portion image in a power-law transform scheme.

The apparatus may further include a contour creator for presenting a contour that represents an area with a suspected lesion using differences in signal intensity between neighboring pixels within the image for diagnosis.

In accordance with another aspect of the present disclosure, a method for detecting a brain lesion in a magnetic resonance (MR) image is provided. The method includes receiving an MR image and creating an image of a brain portion (brain portion image) in which the brain portion is selectively represented; and receiving the brain portion image and performing contrast adjustment on the brain portion image to obtain an image for diagnosis in which an area with a suspected lesion is emphasized.

Creating a brain portion image may include receiving the MR image, and selecting pixels having signal intensities that exceed a standard deviation of signal intensities of all pixels within the MR image for a brain portion; wavelet-transforming the brain portion selected by the binarization transformer to create an image for adjustment; processing the image for adjustment in a quick hull scheme to create a convex image for adjustment; and obtaining the brain portion image by applying the convex image for adjustment to the MR image.

The contrast adjustment may adjust the brain portion image in a power-law transform scheme.

The method may further include presenting a contour that represents an area with a suspected lesion using differences in signal intensity between neighboring pixels within the image for diagnosis.

In accordance with another aspect of the present disclosure, a computer-readable recording medium having a program embodied thereon to carry out the method for detecting a brain lesion in an MR image.

### BRIEF DESCRIPTION OF THE DRAWINGS

The above and other objects, features and advantages of the present invention will be more clearly understood from the following detailed description taken in conjunction with the accompanying drawings, in which:

FIG. 1 is a block diagram of an apparatus for detecting a brain lesion in a magnetic resonance (MR) image, according to an embodiment of the present disclosure;

FIGS. 2A to 2J show images to be processed by an apparatus for detecting a brain lesion in a an MR image, according to an embodiment of the present disclosure; and

FIG. 3 is a flowchart illustrating a method for detecting a brain lesion in an MR image, according to an embodiment of the present disclosure.

### DESCRIPTION OF THE PREFERRED EMBODIMENTS

Embodiments of the present disclosure will now be described with reference to accompanying drawings. The present disclosure may, however, be embodied in many different forms and should not be construed as limited to the embodiments set forth herein. Features and sizes of the components in the drawings may be exaggerated for clarity, and like reference numerals represent like components throughout the drawings.

Some terms as herein used should be interpreted as follows:

Terms “first”, “second”, etc., are to tell respective components from one another, and the scope of the present disclosure should not be limited to the terms. For example, a first element, component, region, layer or section discussed below could be termed a second element, component, region, layer or section, and vice versa.

When A is said to “be connected” to B, it means to be “directly connected” to B or “electrically connected” to B with C located between A and B. The terms “include” or “comprise” are inclusive or open-ended expressions that do not exclude additional, non-recited elements or method steps.

FIG. 1 is a block diagram of an apparatus for detecting a brain lesion in a magnetic resonance (MR) image, according to an embodiment of the present disclosure. An apparatus for detecting a brain lesion in an MR image may include an image area selector 100, an image processor 200, and a contour creator 300.

The image area selector 100 receives an MR image, creates an image of a brain portion (or brain portion image) that selectively represents a brain portion, and outputs the brain portion image to the image processor 200. The image area selector 100 may include a binarization transformer 110, a wavelet transformer 120, a quick-hull processor 130, and a convex processor 140.

The binarization transformer 110 may receive an MR image, select pixels having signal intensities that exceed an average of signal intensities of all pixels within the MR image for a brain portion, and output an image having the brain portion distinguished from other portions through binarization transform to the wavelet transformer 120. Operation of the binarization transformer 110 will now be described in more detail.

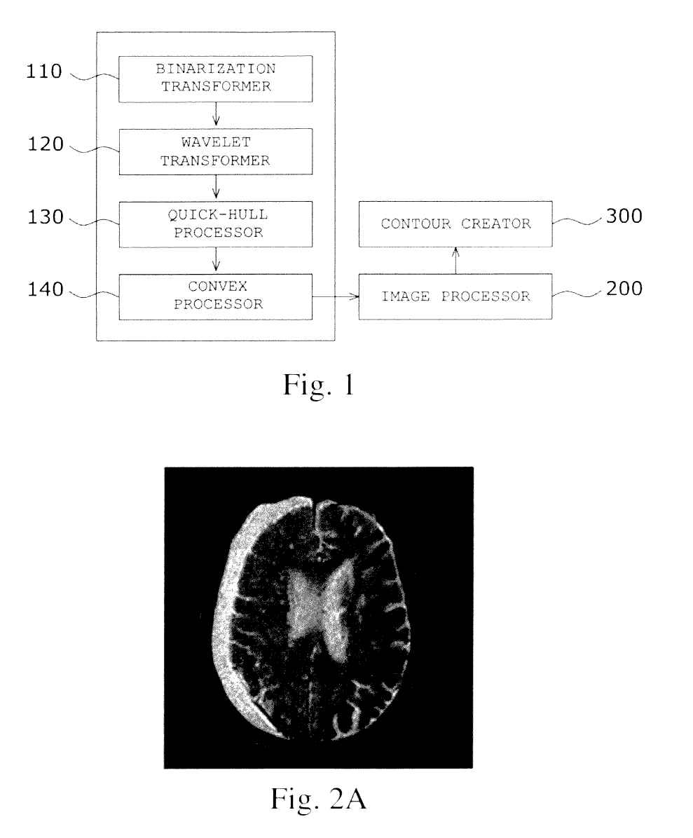

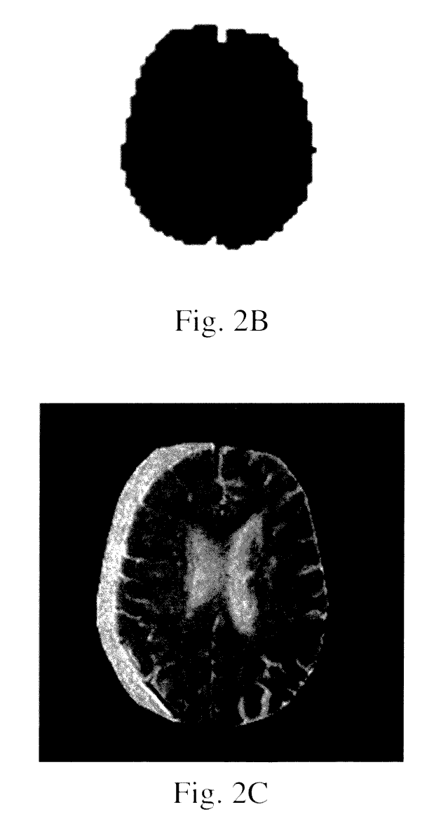

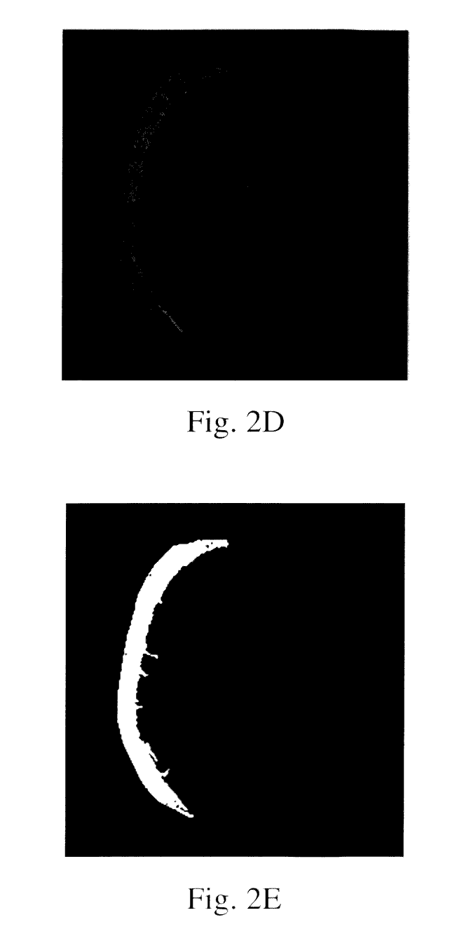

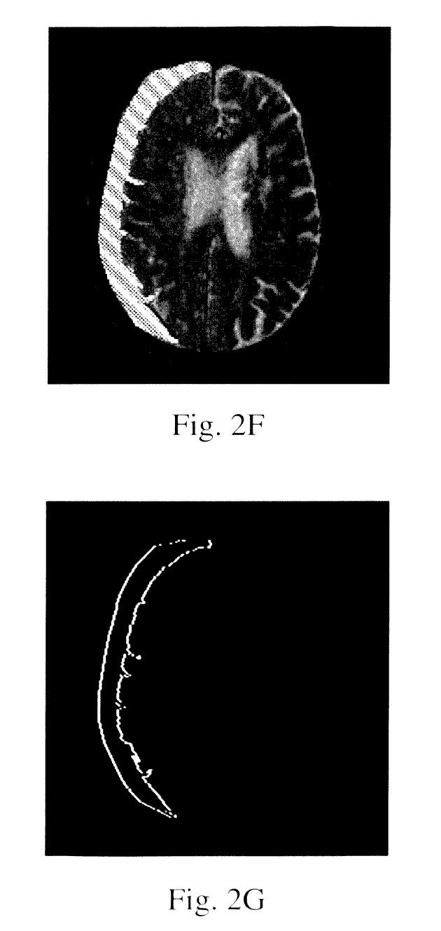

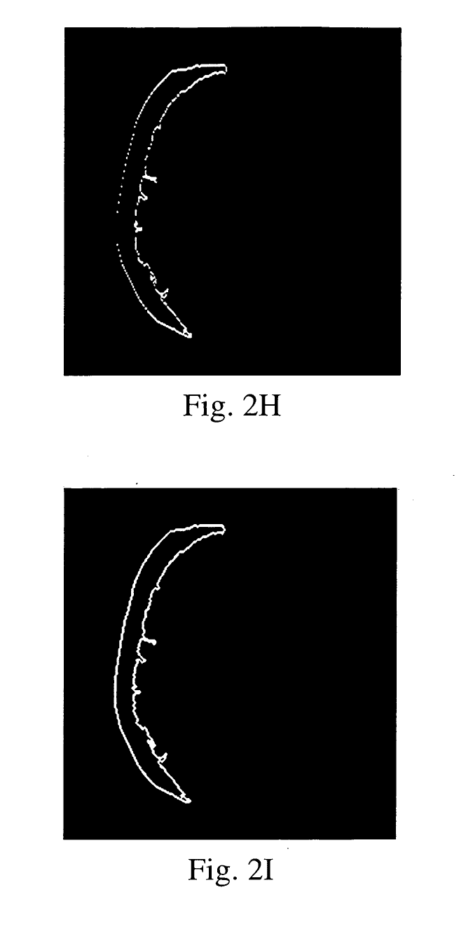

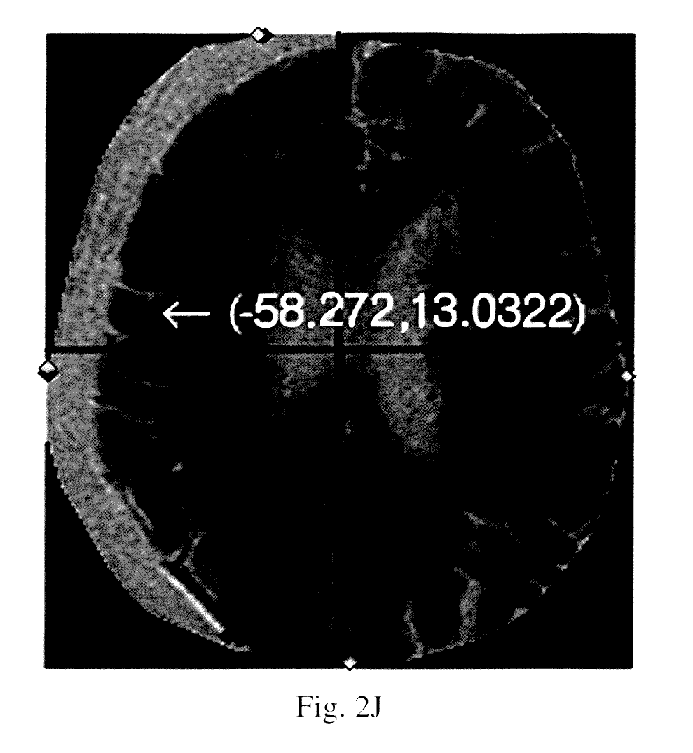

First, given that an intensity value of a signal of a pixel in XY coordinates within an MR image is denoted by f(x, y), the input MR image may be represented by f(x, y), and FIG. 2A shows an example of such an MR image.

Furthermore, a brain portion in the MR image has intensity values of signals distinctly differentiated from other portions, so the brain portion may be relatively accurately extracted through binarization of the MR image using standard deviation σ.

In other words, given that an image having a distinguished brain portion through binarization transform is denoted by f1(x,y), the image may be calculated as in the following equation 1:

$$f 1 ( x , y ) = \{ \begin{matrix}1 & if f ( x , y ) > σ \\ 0 & if f ( x , y ) \leq σ\end{matrix} \qquad ( 1 )$$

To obtain the standard deviation σ, a mean μ is first obtained by dividing a sum of all data coordinates by the number of all coordinates, which is expressed in the following equation 2:

$$μ = \frac{1}{MN} {\sum }_{x = 0}^{M - 1} {\sum }_{y = 0}^{N - 1} f ( x , y ) \qquad ( 2 )$$

where M is the number of samples counted in the X-coordinate direction of a selected image, and N is the number of samples counted in the Y-coordinate direction of the selected image.

Variance v is calculated in the following equation 3:

$$v = \frac{1}{MN} {\sum }_{x = 0}^{M - 1} {\sum }_{y = 0}^{N - 1} {( f ( x , y ) - μ )}^{2} \qquad ( 3 )$$

where, the standard deviation σ, as is well known, equals the square root of the variance.

The standard deviation obtained as described above is used as a threshold to binarize an MR image of the brain, according to which the binarization transformer 110 may be able to extract the brain portion in the MR image and differentiate it from other portions. In other words, the binarization transformer 110 uses the standard deviation as a threshold for signal intensity while performing pre-processing on the MR image.

The wavelet transformer 120 may create an image for adjustment by wavelet-transforming an area selected by the binarization transformer 110, and output the image for adjustment to the quick-hull processor 130. Operation of the wavelet transformer 120 will now be described in more detail.

First, for convenience of the wavelet transform operation, a complementary image f2′(x,y) to the image f1(x,y) having the differentiated brain portion is created as in the following equation 4:

f2′(x,y)=1−f1(x,y) (4)

Like f1(x,y), f2′(x,y) also denotes a binarized image. Subsequently, for function f2′(x) contained in a square integrable function space L²(R), wavelet and scaling functions ψ(x) and φ(x), respectively, may be defined. In other words, in order to increase visibility of an image, wavelet coding is performed on f2′(x), as in the following equation 5:

$$f {2}^{′} ( x ) = {\sum }_{k} {c}_{j 0} ( k ) {φ}_{j 0} ( x ) + {\sum }_{j = j 0}^{∞} {\sum }_{k} {d}_{j} ( k ) {ψ}_{j , k} ( x ) \qquad ( 5 )$$

where, j₀ is an arbitrary initial scale value, cⱼ_{₀}(k) is an approximation coefficient, and dⱼ(k) is a detail coefficient. j determines a resolution level, the first term of the equation 5 including the approximation coefficient refers to an approximation value of the image at resolution level j₀, and the second term including the detail coefficient at each resolution level of j≧j₀ is added to the approximation value to increase details.

In other words, it provides an effect as if an image binarized by the wavelet function has passed a high-pass filter, and an effect as if an image binarized by the scaling function has passed a low-pass filter.

In order to expand the wavelet transform as in the equation 5 into two dimensions, one 2D scaling function and three 2D wavelet functions are required, which may be expressed in the following equations:

φ(x,y)=φ(x)φ(y) (6)

ψ^{H}(x,y)=ψ(x)ψ(y) (7)

ψ^{V}(x,y)=ψ(x)ψ(y) (8)

ψ^{D}(x,y)=ψ(x)ψ(y) (9)

where H refers to a change in the column direction, V refers to a change in the row direction, and D refers to a change in the diagonal direction.

The scaling function and wavelet function at resolution level j, and point (m, n) of the pixel sampled along the x and y coordinates by applying the equations 6 to 9 are denoted by the following equations:

$$\begin{matrix}{ϕ}_{j , m , n} ( x , y ) = {2}^{\frac{j}{2}} ϕ ( {2}^{j} x - m , {2}^{j} y - n ) & ( 10 ) \\ {ψ}_{j , m , n}^{i} ( x , y ) = {2}^{\frac{j}{2}} {ψ}^{i} ( {2}^{j} x - m , {2}^{j} y - n ) & ( 11 )\end{matrix}$$

where i may be selected from a set of {H, V, D}.

For image f2′(x, y) having a size of M×N, a wavelet expansion function is denoted by the following equations:

$$\begin{matrix}{W}_{ϕ} ( {j}_{0} , m , n ) = \frac{1}{\sqrt{MN}} {\sum }_{x = 0}^{M - 1} {\sum }_{y = 0}^{N - 1} f {2}^{′} ( x , y ) {ϕ}_{{j}_{0 ,} m , n} ( x , y ) & ( 12 ) \\ {W}_{{ψ}^{i}} ( j , m , n ) = \frac{1}{\sqrt{MN}} {\sum }_{x = 0}^{M - 1} {\sum }_{y = 0}^{N - 1} f {2}^{′} ( x , y ) {ψ}_{{j}_{,} m , n}^{i} ( x , y ) & ( 13 )\end{matrix}$$

Accordingly, an image for adjustment, f2(x,y), created by applying the wavelet expansion function according to the equations 12 and 13 is denoted by the following equation 14:

$$f 2 ( x , y ) = \frac{1}{\sqrt{MN}} {\sum }_{m} {\sum }_{n} {W}_{ϕ} ( {j}_{0} , m , n ) {ϕ}_{{j}_{0} , m , n} ( x , y ) + \frac{1}{\sqrt{MN}} {\sum }_{i = H , V , D}^{ } {\sum }_{j = {j}_{0}}^{∞} {\sum }_{m}^{ } {\sum }_{n}^{ } {W}_{{ψ}^{i}} ( j , m , n ) {ψ}_{{j}_{,} m , n}^{i} ( x , y ) \qquad ( 14 )$$

Consequently, the wavelet transformer 120 may obtain an image for adjustment in which a brain portion and non-brain portion are distinctly distinguished from each other through the above operations, and an example of the image for adjustment is shown in FIG. 2B.

The quick-hull processor 130 may receive the image for adjustment from the wavelet transformer 120, process the received image in a quick-hull scheme to create a convex image for adjustment, and output the convex image to the convex processor 140. Specifically, the quick-hull processor 130 may obtain a convex hull including all the pixels classified as the brain portion in the image for adjustment as shown in FIG. 2B through a divide-and-conquer algorithm, and the convex hull corresponds to convex edges containing the convex image for adjustment, i.e., the entire brain portion.

The quick-hull processor 130 may store the obtained convex image for adjustment, and reuse the stored convex image over and over again for subsequently input MR images.

The convex processor 140 may receive the convex image for adjustment from the quick-hull processor 130, create a brain portion image f3(x, y) by applying the convex image to an MR image, and output the created brain portion image to the image processor 200. Specifically, the convex processor 140 may create the brain portion image by eliminating the convex image created by the quick-hull processor 130, i.e., the outside area of the convex edges, from the MR image as shown in FIG. 2A, and an example of the brain portion image is shown in FIG. 2C.

More specifically, the convex processor 140 may create an image with artifacts, the skull, etc., eliminated by eliminating the outside area of the convex edges from the MR image, and by doing this, unnecessary portions that might be detected as an abnormal portion in making a diagnosis may be removed.

The image processor 200 may obtain an image for diagnosis, f4(x,y), in which an area with a suspected lesion is emphasized, by performing contrast adjustment on the brain portion image received from the convex processor 140 of the image area selector 100. The image processor 200 may output the obtained image for diagnosis to the contour creator 300. The operation for performing contrast adjustment on the brain portion image in the image processor 200 may be performed by simply adjusting contrast values of the image, but more preferably, performed by the following power-law transform method.

First, the image processor 200 may adjust an image through a power-law transform method as denoted in the following equation 15:

f4(x,y)c×(f3(x,y)+ε)^{≡} (15)

where, ε corresponds to an offset, which is a value that enables measurement even for an input image with all the pixels of zero value. A transform effect in a case that a gamma value γ is greater than 1 and a transform effect in a case that a gamma value γ is smaller than 1 are opposite. If c=γ=1, an effect of the identity transformation is gained. The gamma value has an influence not only on the signal intensity but also proportions of RGB (Red, Green, and Blue) signals, so an image good for distinction of a lesion portion may be obtained through appropriate adjustment of the gamma value. In the meantime, an example of an image f4(x,y) created by processing the brain portion image in the power-law transform method is shown in FIG. 2D.

The final intensity of signals of the power-law transformed image obtained according to the equation 15 is derived by the following equation 16:

$$T = \frac{1}{MN} {\sum }_{x = 0}^{M - 1} {\sum }_{y = 0}^{N - 1} f 4 ( x , y ) + \sqrt{\frac{1}{MN} {\sum }_{x = 0}^{M - 1} {\sum }_{y = 0}^{N - 1} {( f 4 ( x , y ) - μ )}^{2}} \qquad ( 16 )$$

An image f5(x, y) as shown in FIG. 2E in which only a portion suspected with a lesion is presented may be obtained by performing binarization transform on the image f4(x, y) based on the final signal intensity T calculated according to the equation 16, and an image for diagnosis as shown in FIG. 2F may be obtained e.g., by multiplying image that represents the area with a suspected lesion by pixel values corresponding to the MR image.

The contour creator 300 may receive the image for diagnosis from the image processor 200, and present a contour that represents an area with a suspected lesion using the difference in intensity between signals of neighboring pixels within the image for diagnosis. The contour creator 300 may create the contour using a binarization transformed image as shown in FIG. 2E instead of the image for diagnosis, and in this case, contours in the horizontal and vertical directions as shown in FIGS. 2G and 2H may be composed by the following equations 17 and 18, respectively:

$$\begin{matrix}{h}_{c} = \frac{\partial f}{\partial x} = f 5 ( x + 1 ) - f 5 ( x ) & ( 17 ) \\ {v}_{c} = \frac{\partial f}{\partial y} = f 5 ( y + 1 ) - f 5 ( y ) & ( 18 )\end{matrix}$$

The contours composed by the equations 17 and 18 are discontinuous. A continuous contour may be obtained as shown in FIG. 2I by combining the contours in the horizontal and vertical directions according to the following equation 19.

c_{c}=h_{c}+v_{c} (19)

To extract a location of the portion with a suspected lesion, measurement of a center of the portion of e.g., apoplexy is required, and a type of the lesion may be identified through the measurement. For this, coordinate values of the center may be obtained after weights I₁˜Iₚ are given to the respective coordinates as denoted in the following equation 20, and a result of marking the center on the image of FIG. 2C is shown in FIG. 2J.

X_{cood}=Σₙ₌₁^{p}xₙIₙ/Σₙ₌₁^{p}Iₙ

Y_{cood}=Σₙ₌₁^{p}yₙIₙ/Σₙ₌₁^{p}Iₙ (20)

For reference, to distinguish a brain lesion image as shown in FIG. 2A, a method for transforming the MR image, which is an RGB image, to a grayscale image may be used. The image transform is performed by issuing weights by multiplying R, G, and B components by respective constants, and summing the weighted values. This transform is well known to ordinary people skilled in the art, so the description is omitted herein for convenience of explanation.

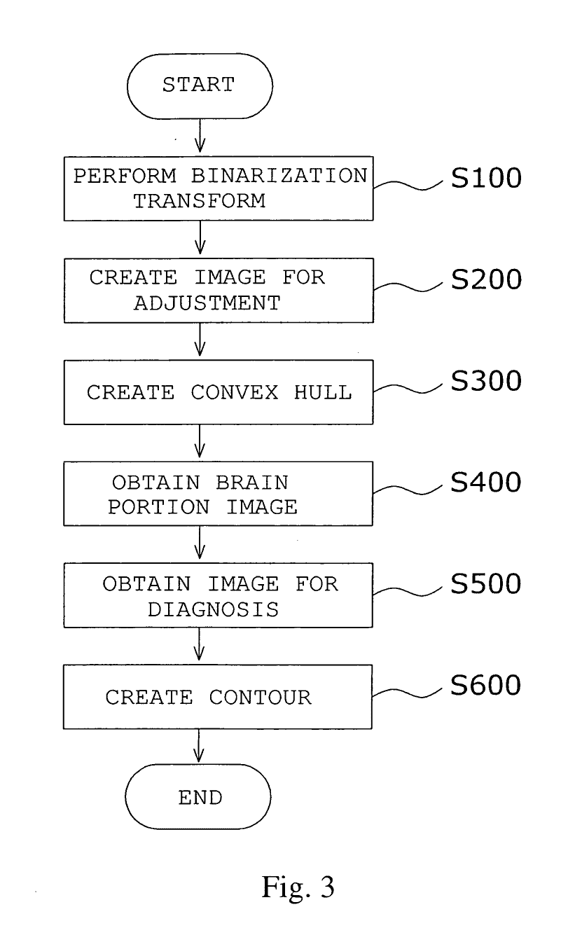

FIG. 3 is a flowchart illustrating a method for detecting a brain lesion in an MR image, according to an embodiment of the present disclosure. Referring to FIGS. 1 to 3, a method for detecting a brain lesion in an MR image in accordance with an embodiment of the present disclosure will be described below.

First, an MR image is received to create an image of a brain portion (or brain portion image) in which a brain portion is selectively represented. This will be described below in detail.

Upon reception of an MR image, pixels having signal intensities that exceed a standard deviation of signal intensities of all pixels within the MR image are selected for a brain portion, in operation S100. A pixel is allocated a value ‘1’ or ‘0’ according to whether the pixel belongs to the brain portion, which equals to binarization transform on the MR image.

Next, an image for adjustment is created by wavelet transform on a selected area, in operation S200. Through the wavelet transform, filtering is performed on the binarized image to select a more accurate brain portion.

Subsequently, the image for adjustment is processed into a convex image for adjustment in a quick-hull method, in operation S300. Specifically, a convex hull enclosing an area distinguished by the wavelet transform as the brain portion, i.e., convex edge is created.

Next, a brain portion image is obtained by applying the convex image for adjustment to the MR image, in operation S400. This enables obtaining an image of the brain portion, from which the skull or artifact present in the outside of the convex image is eliminated.

Subsequently, an image for diagnosis in which an area with a suspected lesion is emphasized is obtained by performing contrast adjustment on the received brain portion image, in operation S500. In the contrast adjustment, an area with a suspected lesion may be emphasized by setting power series to be smaller or greater than ‘1’ in advance based on characteristics of each lesion in a power-law transform method.

A contour representing the area with the suspected region is created using the difference in signal intensity between neighboring pixels within the image for diagnosis, in operation S600. Alternatively, the contour may be created using the binarized image of FIG. 2E instead of the image for diagnosis.

The method for detecting a lesion in an MR image in accordance with embodiments of the present disclosure may be implemented by a program that is recorded in a computer-readable recording medium, e.g., Compact Disc Read Only Memory (CD-ROM), Random Access Memory (RAM), ROM, floppy disk, hard disk, magneto-optical disc, etc.

According to embodiments of the present disclosure, a user of an MRI device may selectively read an image required, by providing an MR image that selectively represents a brain portion in making a diagnosis of a lesion or hemorrhage due to e.g., apoplexy.

Furthermore, with a function to perform power-law transform on an MR image so as to emphasize distinction of a lesion within the MR image that represent a brain portion, an image with a lesion portion emphasized may be easily obtained by the user properly adjusting power series of the power-low transform function.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

What is claimed is:

1. An apparatus for detecting a brain lesion in a magnetic resonance (MR) image, the apparatus comprising:
an image area selector for receiving an MR image and creating an image of a brain portion (brain portion image) in which the brain portion is selectively presented; and
an image processor for receiving the brain portion image and performing contrast adjustment on the brain portion image to obtain an image for diagnosis in which an area with a suspected lesion is emphasized.

2. The apparatus of claim 1, wherein the image area selector comprises
a binarization transformer for receiving the MR image, and selecting pixels, having signal intensities that exceed a standard deviation of signal intensities of all pixels within the MR image for a brain portion;
a wavelet transformer for wavelet-transforming the brain portion selected by the binarization transformer to create an image for adjustment;
a quick-hull processor for processing the image for adjustment in a quick hull scheme to create a convex image for adjustment; and
a convex processor for obtaining the brain portion image by applying the convex image for adjustment to the MR image.

3. The apparatus of claim 1, wherein the contrast adjustment is to adjust the brain portion image in a power-law transform scheme.

4. The apparatus of claim 1, further comprising: a contour creator for presenting a contour that represents an area with a suspected lesion using differences in signal intensity between neighboring pixels within the image for diagnosis.
